(19) United States
(12) Patent Application Publication
(10) Pub. No.: US 2017/0165186 A1
(43) Pub. Date: Jun. 15, 2017

(54) COSMETIC COMPOSITION FOR TEMPORARY RESHAPING OF KERATINIC FIBERS

(71) Applicant: Henkel AG & Co. KGaA, Duesseldorf (DE)

(72) Inventors: Julia Bibiane Lange, Bad Bramstedt (DE); Bernd Richters, Hamburg (DE); Maria Catalina Bermudez Agudelo, Hamburg (DE); Cyrielle Martinez, Hamburg (DE)

(73) Assignee: Henkel AG & Co. KGaA, Duesseldorf (DE)

(21) Appl. No.: 15/444,483

(22) Filed: Feb. 28, 2017

Related U.S. Application Data
(63) Continuation of application No. PCT/EP2015/064653, filed on Jun. 29, 2015.

(30) Foreign Application Priority Data
Aug. 28, 2014 (DE) 10 2014 217 206.3

(51) Int. Cl. A61K 8/81; A61Q 5/06

(52) U.S. Cl. CPC A61K 8/8176; A61Q 5/06; A61K 8/8152; A61K 2800/48; A61K 2800/594

(57) ABSTRACT

The invention relates to a cosmetic composition for the temporary shaping of hair, including a combination of the two specific (co)polymers. Said cosmetic preparation provides an extremely good combination of stiffness and long-term hold.

### FIELD OF THE INVENTION

The present invention relates to a cosmetic composition for fixing hair or for the temporary reshaping of keratin fibers, in particular human hair, wherein the composition includes a combination of an anionic acrylate resin and a vinylpyrrolidone homopolymer.

### BACKGROUND OF THE INVENTION

The temporary styling of hairstyles for a longer period up to a number of days generally requires the application of active fixing agents. Hair treatment agents which serve to temporarily shape the hair therefore play a key role. Suitable agents for temporary shaping usually include synthetic polymers and/or waxes as active fixing agent. Agents for assisting the temporary reshaping of keratin-containing fibers can be formulated for example as a hairspray, hair wax, hair gel or hair mousse.

The most important property of an agent for temporarily shaping hair, also referred to hereinafter as styling agents, lies in providing the treated fibers in the newly modelled form—i.e. a form impressed onto the hair—with the strongest hold possible. Reference is also made to a strong hairstyle hold or a high degree of hold of the styling agent. The hairstyle hold is determined substantially by the type and amount of the active fixing agents used, wherein the further constituents of the styling agent however can also make a contribution.

Besides a high degree of hold, it can be desirable that the styling agents meet a wide range of further requirements. These can be divided roughly into properties on the hair, properties of the particular formulation, for example properties of the mousse, the gel, or the sprayed aerosol, and properties which concern the handling of the styling agent, wherein the properties on the hair are attributed particular importance. The following should be mentioned in particular: moisture resistance, low tack, and a balanced conditioning effect. Furthermore, a styling agent should be universally usable for all hair types where possible and should be mild on the hair and skin.

In order to meet the different requirements, a large number of synthetic polymers have already been developed as active fixing agents and are used in styling agents. The polymers can be divided into cationic, anionic, non-ionic and amphoteric fixing polymers. When applied to the hair, the polymers ideally provide a polymer film which on the one hand provides the hairstyle with a strong hold, but on the other hand is sufficiently flexible so as not to break under stress. If the polymer film is too fragile, what are known as film flakes form, i.e. residues, which come loose as the hair moves and give the impression that the user of the corresponding styling agent has dandruff. Similar problems occur when waxes are used as active fixing agent in the styling agent. If the styling agent is a gel or a paste, the polymers additionally have thickening properties.

Known anionic polymers that can be used in hair fixing products are hydrophobically modified acrylate copolymers. These are commercially available (INCI: Acrylates Copolymer (and) Water) and act fundamentally as thickening agent. The datasheet AquaStyle® SH-100 Polymer (Ashland Inc.) describes such an acrylate copolymer and use thereof in combination with carbomers. A suitability for crystal-clear hair gels, good initial stiffness, moisture resistance and durable effect is described.

Known non-ionic polymers which are used in hair fixing products are polyvinylpyrrolidones (PVP). These are homopolymers of vinylpyrrolidone. Vinylpyrrolidone homopolymers are sold for example under the name Luviskol® (BASF) and are usually used as film formers and/or thickening agents. The product range Luviskol® K (BASF) is obtainable in particular for the use in cosmetic compositions and styling products as film formers or thickening agents and provides vinylpyrrolidone homopolymers in various molecular weights.

The object of the present invention was to provide further suitable polymer combinations which are characterized by good film-forming and/or fixing properties, have a high degree of hold without having to compromise on flexibility or good moisture resistance—in particular resistance to perspiration and water—and additionally are suitable for the production of stable viscous and also stable transparent cosmetic compositions. In particular, currently obtainable styling agents can be improved yet further because a good combination of stiffness and long-term hold is not always sufficiently ensured. The object of the present invention is therefore to provide such styling agents which, in addition to the above-mentioned properties, provide in particular both good stiffness and good long-term hold.

### BRIEF SUMMARY OF THE INVENTION

The following is provided by the present invention:
1. A cosmetic composition for temporarily reshaping keratin fibers, said composition comprising:
   (a) a vinylpyrrolidone homopolymer and,
   (b) at least one anionic acrylate copolymer (b) which is constructed at least from the following monomer units: at least one (meth) acrylic acid unit (b1), at least one (meth)acrylic acid ethyl ester unit (b2), and at least one (meth)acrylic acid ester unit (b3) which is different from the (meth)acrylic acid ethyl ester unit (b2) and comprises a hydrophobic group as ester group.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

This has been achieved in accordance with the invention by a combination of a specific anionic acrylate resin and a non-ionic polymer.

Within the scope of the present invention, it has been surprisingly found that by combining two polymers known per se, an improved combination of long-term effect and stiffness of styling products, in particular of hair gels, can be obtained. A combination of this type of good stiffness and good long-term hold was not expected.

The term keratin fibers includes fur, wool and feathers in accordance with the invention, but in particular human hair.

The essential constituents of the cosmetic composition according to the invention are the vinylpyrrolidone homopolymer (a) and the anionic acrylate copolymer (b).

The agent according to the invention mandatorily includes, as component (a), a vinylpyrrolidone homopolymer. It is preferred in accordance with the invention to select the vinylpyrrolidone homopolymers from vinylpyrrolidone homopolymers having a K value (1% by weight solution of PVP, Brookfield at 23° C.) in water of from 20 to 100. What is more preferred is a K value of from 80 to 100, more preferably approximately 90. The K value, referred to as inherent viscosity, is a parameter for the characterization of polymers that is easily determined from the relative viscosity by means of viscosity measurements of polymer solutions.

Preferred vinylpyrrolidone homopolymers are obtainable under the trade names Luviskol® K30, Luviskol® K80, Luviskol® K85, Luviskol® K90, in each case from the company BASF SE. In accordance with the invention, Luviskol® K90 is most preferred. Luviskol® K90 is a 20% aqueous, colorless to light yellow solution of polyvinylpyrrolidone. The product has a K value of from 90.0 to 98.0 (1% (m/V) in water), a solids content of from 19.0 to 21.0% by weight, and a pH value of from 7.0 to 9.0 (10% by weight solids content in water).

The anionic acrylate copolymer (b) is constructed at least from the following monomer units: at least one (meth) acrylic acid unit (b1), at least one (meth) acrylic acid ethyl ester unit (b2), and at least one (meth) acrylic acid ester unit (b3) which is different from the (meth) acrylic acid ethyl ester unit (b2) and comprises a hydrophobic group as ester group.

The copolymer (b) can be constructed in accordance with the invention from further monomer units. In accordance with embodiments of the invention, the copolymer (a), however, is constructed only from the units (b1), (b2) and (b3), i.e. it consists of units derived from these monomer units.

The at least one (meth)acrylic acid unit (b1) can be a meth acrylic acid unit or acrylic acid unit, wherein a meth acrylic acid unit is preferred.

The at least one (meth)acrylic acid ethyl ester unit (b2) can be a meth acrylic acid ethyl ester unit or an acrylic acid ethyl ester unit, wherein an acrylic acid ethyl ester unit is preferred.

The at least one (meth) acrylic acid ester unit (b3) can be a (meth) acrylic acid alkyl ester unit in accordance with the invention. The alkyl group of the (meth) acrylic acid alkyl ester unit serves to control the hydrophobicity of the copolymer. The alkyl group is preferably a linear or branched alkyl group with 2 to 30 carbon atoms, more preferably 3 to 12 carbon atoms. In accordance with the invention, the hydrophobic group can also be a hydrophobic group other than an alkyl group, for example an aromatic hydrocarbon ester group. A substituted or unsubstituted phenyl ester group or substituted or unsubstituted alkylene phenyl ester group can be cited as examples, for example a benzyl ester group.

The viscosity of the anionic acrylate copolymer (b) used in the cosmetic composition, with a solids content of 2% by weight and neutralized solution at 25° C., is preferably at most 60,000 to 120,000 CPS.

Suitable anionic acrylate copolymers (b) are commercially available under the INCI name Acrylates Copolymer (and) Water.

The anionic acrylate copolymer (b) AquaStyle® SH-100 polymer from Ashland, Inc. is most preferred. In the commercially available form, this has a solids content of approximately 28 to 32% by weight and a pH value of 2.1-4.0.

The cosmetic composition of the present invention includes the vinylpyrrolidone homopolymer (a) and acrylate copolymer (b) in amounts usual and suitable for styling agents, which amounts can be adapted to the specific application and formulation.

The cosmetic composition according to the invention includes the vinylpyrrolidone homopolymer (a), in relation to the total weight of the cosmetic composition, for example in an amount of from 0.1 to 5% by weight, preferably 0.1 to 1.5% by weight, more preferably 0.2 to 0.8% by weight, specified in each case as solids content of active substance in the cosmetic composition.

The cosmetic composition according to the invention includes the acrylate copolymer (b), in relation to the total weight of the cosmetic composition, for example in an amount of from 0.1 to 5% by weight, preferably 0.3 to 2.0% by weight, more preferably 0.8 to 1.4% by weight, specified in each case as solids content of active substance in the cosmetic composition.

In a particularly preferred embodiment of the present invention, the cosmetic composition includes, as the vinylpyrrolidone homopolymer (a), the copolymer obtainable under the name Luviskol® K90 (BASF), and, as the anionic acrylate copolymer (b), the copolymer commercially available under the name AquaStyle™ SH-100. In the case of this combination, particularly good results in terms of a combination of stiffness and long-term hold were attained. This polymer combination is particularly advantageous in the case of styling products in gel form.

Further generally required properties of styling products, such as resistance to moisture and low tack, are attained equally in particular with this combination, particularly when the product is formulated as a hair gel.

The cosmetic composition of the present invention preferably includes one or more further component(s) acting as thickening agent or gel fomer which is/are different from the vinylpyrrolidone homopolymer (a) and the acrylate copolymer (b) and likewise assist the film formation. Examples are cationic, anionic, non-ionic or amphoteric polymers. The proportion by weight of these further components in the total weight of the cosmetic composition can be relatively low on account of the presence of the components (a) and (b) and for example is 0.02 to 3% by weight, preferably 0.05 to 1.5% by weight, and even more preferably 0.2 to 0.8% by weight.

Examples are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates/CI-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylat.es/C1-18 Alkyl Acrylates/C 1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Com Starch/Acrylamide/ Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolysed Wheat Protein/PVP Crosspolymer, Isobutylene/Ethylmaleimide/Hydroxyethylmaleimide Copolymer, Isobutyl ene/MA Copolymer, Isobutyl methacryl ate/B i s-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethyl acrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/ltaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene VP Copolymer.

Examples of non-ionic polymers are:
Vinylpyrrolidone/vinyl ester copolymers, as are sold for example under the trade name Luviskol (BASF). Luviskol VA 64 and Luviskol VA 73, each vinylpyrrolidone/vinyl acetate copolymers, are preferred non-ionic polymers.
Cellulose ethers, such as hydroxypropyl cellulose, hydroxyethyl cellulose and methylhydroxypropyl cellullose, as are sold for example under the trade names Culminal and Benecel (AQUALON)
Shellac.
Siloxanes. These siloxanes can be both water-soluble and water-insoluble. Both volatile and non-volatile siloxanes are suitable, wherein compounds of which the boiling point at normal pressure is above 200° C. are understood to be non-volatile siloxanes. Preferred siloxanes are polydialkylsiloxanes, such as polydimethyl siloxane, polyalkylaryl siloxanes, such as polyphenylmethyl siloxane, ethoxylated polydialkyl siloxanes, and polydialkyl siloxanes which include amine and/or hydroxy groups.
Glycosidically substituted silicones.

The further component acting as gel former is preferably a homopolyacryllic acid Carbomer), which is commercially available under the name Carbopol® in various embodiments. The carbomer is preferably conincludedtained in a proportion of from 0.02 to 3% by weight, preferably 0.05 to 1.5% by weight, and even more preferably 0.2 to 0.8% by weight, in relation to the total weight of the cosmetic composition.

The cosmetic composition can include further conventional substances of styling products. In particular, additional nourishing ingredients can be mentioned as further auxiliaries and additives.

As nourishing ingredient, the agent can include at least one protein hydrolysate and/or a derivative thereof, for example. Protein hydrolysates are product mixtures which are obtained by acid-catalysed, base-catalysed or enzymatically catalysed breakdown of proteins. The term “protein hydrolysates” is understood in accordance with the invention to also mean total hydrolysates and also individual amino acids and derivatives thereof as well as mixtures of different amino acids. The molecular weight of the protein hydrolysates usable in accordance with the invention lies between 75, the molecular weight for glycine, and 200,000 daltons, and the molecular weight is preferably 75 to 50,000 daltons, and very particularly preferably 75 to 20,000 daltons.

As nourishing ingredient, the agent according to the invention can also include at least one vitamin, a provitamin, a vitamin precursor and/or one of the derivatives thereof. Here, vitamins, provitamins and vitamin precursors that are usually assigned to the groups A, B, C, E, F and H are preferred in accordance with the invention.

Similarly to the addition of glycerol and/or propylene glycol, the addition of panthenol increases the flexibility of the polymer film formed with application of the agent according to the invention.

As nourishing ingredient, the agents according to the invention can also include at least one plant extract, but also monosaccharides or oligosaccharides and/or lipids.

Oil bodies are also suitable as nourishing ingredient. Natural and synthetic cosmetic oil bodies include, for example, plant oils, liquid paraffin oils, isoparaffin oils and synthetic hydrocarbons as well as di-n-alkyl ethers having a total of between 12 and 36 C atoms, in particular 12 to 24 C atoms. Preferred cosmetic agents according to the invention include at least one oil body, preferably at least one oil body from the group of silicone oils. The group of silicone oils includes, in particular, the dimethicones, which also include the cyclomethicones, the aminofunctional silicones, and also the dimethiconols. The dimethicones can be both linear and branched and cyclic, or cyclic and branched. Suitable silicone oils or silicone gums are in particular dialkyl and alkylaryl siloxanes, such as dimethyl polysiloxane and methylphenyl polysiloxane, and the alkoxylated, quaternised, or anionic derivatives thereof. Cyclic and linear polydialkyl siloxanes, the alkoxylated and/or aminised derivatives, dihydroxypolydimethyl siloxanes and polyphenyl alkyl siloxanes are preferred.

Ester oils, i.e. esters of C6-C30 fatty acids with C2-C30 fatty alcohols, preferably monoesters of the fatty acids with alcohols having 2 to 24 C atoms, such as isopropylmyristate (Rilanit® IPM), isononanoic acid C16-18 alkyl ester (Cetiol® SN), 2-ethylhexyl palmitate (Cegesoft® 24), stearic acid-2-ethylhexyl ester (Cetiol® 868), cetyl oleate, glycerol tricaprylate, coconut fatty alcohol caprate/caprylate (Cetiol® LC), n-butyl stearate, oleyl erucate (Cetiol® J 600), isopropyl palmitate (Rilanite® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), and oleic acid decyl ester (Cetiol® V) are further preferred nourishing oil bodies.

Furthermore, dicarboxylic acid esters, symmetrical, asymmetrical or cyclic esters of carbon dioxide with fatty alcohols, tri fatty acid esters of saturated and/or unsaturated linear and/or branched fatty acids with glycerol or fatty acid partial glycerides, which are understood to mean monoglycerides, diglycerides and industrial mixtures thereof, are to be understood as nourishing ingredients.

Emulsifiers or surface-active agents are also preferably included in the composition according to the invention. PEG derivatives of hydrogenated castor oil which are obtainable for example under the name PEG Hydrogenated Castor Oil are preferred, for example PEG-30 Hydrogenated Castor Oil, PEG-33 Hydrogenated Castor Oil, PEG-35 Hydrogenated Castor Oil, PEG-36 Hydrogenated Castor Oil, or PEG-40 Hydrogenated Castor Oil. The use of PEG-40 Hydrogenated Castor Oil is preferred in accordance with the invention. These are preferably included in an amount of from 0.05 to 1.5% by weight, more preferably 0.1 to 1.0% by weight, also preferably 0.2 to 0.8% by weight or 0.3 to 0.6% by weight.

The cosmetic agents according to the invention include the ingredients or active substances in a cosmetically acceptable carrier.

Preferred cosmetically acceptable carriers are aqueous, alcoholic, or aqueous-alcoholic media with preferably at least 10% by weight water, calculated on the basis of the total weight of the agent.

The cosmetic carrier particularly preferably includes water, in particular in such an amount that the cosmetic agent, calculated on the basis of the total weight of the agent, includes at least 10% by weight, in particular at least 20.0% by weight, most preferably at least 40% by weight.

As alcohols, the lower alcohols with 1 to 4 carbon atoms usually used in particular for cosmetic purposes, such as ethanol and isopropanol, can be included.

Examples of water-soluble solvents as co-solvents are glycerol and/or ethylene glycol and/or 1,2-propylene glycol in an amount of from 0 to 30% by weight in relation to the total agent.

The cosmetic composition of the present invention can be formulated in the ways usual for the temporary reshaping of hair, for example as a hair gel, hairspray, hair mousse or hair wax. Formulation as a hair gel is preferred.

Both hair mousses and hairsprays require the presence of propellants. In accordance with the invention, however, no hydrocarbons or only small amounts of hydrocarbons should preferably be used for this purpose. Dimethyl ether is a suitable propellant in accordance with the invention.

The present invention also relates to the use of cosmetic compositions according to the invention for temporarily reshaping keratin fibers, in particular human hair, and also a method for temporarily shaping keratin fibers, in particular human hair, in which the cosmetic composition according to the invention is applied to keratin fibers.

Table Overview

The composition of some preferred cosmetic agents can be deduced from the following tables (specified amounts in % by weight in relation to the total weight of the cosmetic agent unless specified otherwise).

| | Formula | Formula | Formula | Formula | Formula |
|---|---|---|---|---|---|
| | 1 | 2 | 3 | 4 | 5 |
| Vinyl- | 0.05 to 3.0 | 0.1 to 2.5 | 0.2 to 2.0 | 0.3 to 1.6 | 0.4 to 1.4 |
| pyrrolidone | | | | | |
| homopolymer | | | | | |
| (a) | | | | | |
| Copolymer (b) | 0.1 to 5.0 | 0.2 to 4.0 | 0.3 to 2.0 | 0.6 to 1.4 | 0.8 to 1.2 |
| Misc | to 100 | to 100 | to 100 | to 100 | to 100 |

| | Formula | Formula | Formula | Formula | Formula |
|---|---|---|---|---|---|
| | 1a | 2a | 3a | 4a | 5a |
| Vinyl- | 0.05 to 3.0 | 0.1 to 2.5 | 0.2 to 2.0 | 0.3 to 1.6 | 0.4 to 1.4 |
| pyrrolidone | | | | | |
| homopolymer | | | | | |
| (a) with K | | | | | |
| value of from | | | | | |
| 80 to 100 | | | | | |
| (specified as | | | | | |
| solids content) | | | | | |
| Copolymer | 0.1 to 5.0 | 0.2 to 4.0 | 0.3 to 2.0 | 0.6 to 1.4 | 0.8 to 1.2 |
| (b): Acrylates | | | | | |
| Copolymer | | | | | |
| (and) Water | | | | | |
| Misc | to 100 | to 100 | to 100 | to 100 | to 100 |

| | Formula | Formula | Formula | Formula | Formula |
|---|---|---|---|---|---|
| | 1b | 2b | 3b | 4b | 5b |
| Vinyl- | 0.05 to 3.0 | 0.1 to 2.5 | 0.2 to 2.0 | 0.3 to 1.6 | 0.4 to 1.4 |
| pyrrolidone | | | | | |
| homopolymer | | | | | |
| (a): | | | | | |
| Luviskol ® | | | | | |
| K 90-20% | | | | | |
| (specified as | | | | | |
| solids content) | | | | | |
| Copolymer | 0.1 to 5.0 | 0.2 to 4.0 | 0.3 to 2.0 | 0.6 to 1.4 | 0.8 to 1.2 |
| (b): | | | | | |
| AquaStyle ® | | | | | |
| SH-100 | | | | | |
| (specified as | | | | | |
| solids content) | | | | | |
| Misc | to 100 | to 100 | to 100 | to 100 | to 100 |

| | Formula | Formula | Formula | Formula | Formula |
|---|---|---|---|---|---|
| | 1 | 2 | 3 | 4 | 5 |
| Vinyl- | 0.05 to 3.0 | 0.1 to 2.5 | 0.2 to 2.0 | 0.3 to 1.6 | 0.4 to 1.4 |
| pyrrolidone | | | | | |
| homo- | | | | | |
| polymer | | | | | |
| (a) | | | | | |
| Copolymer | 0.1 to 5.0 | 0.2 to 4.0 | 0.3 to 2.0 | 0.6 to 1.4 | 0.8 to 1.2 |
| (b) | | | | | |
| Carbomer | 0.02 to 3.0 | 0.05 to 2.0 | 0.05 to 1.5 | 0.2 to 1.0 | 0.35 to 0.8 |
| Misc | to 100 | to 100 | to 100 | to 100 | to 100 |

| | Formula | Formula | Formula | Formula | Formula |
|---|---|---|---|---|---|
| | 11a | 12a | 13a | 14a | 15a |
| Vinyl- | 0.05 to 3.0 | 0.1 to 2.5 | 0.2 to 2.0 | 0.3 to 1.6 | 0.4 to 1.4 |
| pyrrolidone | | | | | |
| homo- | | | | | |
| polymer | | | | | |
| (a) with K | | | | | |
| value of from | | | | | |
| 80 to100 | | | | | |
| (specified as | | | | | |
| solids | | | | | |
| content) | | | | | |
| Copolymer | 0.1 to 5.0 | 0.2 to 4.0 | 0.3 to 2.0 | 0.6 to 1.4 | 0.8 to 1.2 |
| (b): | | | | | |
| Acrylates | | | | | |
| Copolymer | | | | | |
| (and) Water | | | | | |
| Carbomer | 0.02 to 3.0 | 0.05 to 2.0 | 0.05 to 1.5 | 0.2 to 1.0 | 0.35 to 0.8 |
| Misc | to 100 | to 100 | to 100 | to 100 | to 100 |

| | Formula | Formula | Formula | Formula | Formula |
|---|---|---|---|---|---|
| | 11b | 12b | 13b | 14b | 15b |
| Vinyl- | 0.05 to 3.0 | 0.1 to 2.5 | 0.2 to 2.0 | 0.3 to 1.6 | 0.4 to 1.4 |
| pyrroli- | | | | | |
| done | | | | | |
| homo- | | | | | |
| polymer | | | | | |
| (a): | | | | | |
| Luviskol ® | | | | | |
| K 90-20% | | | | | |
| (specified | | | | | |
| as solids | | | | | |
| content) | | | | | |
| Copolymer | 0.1 to 5.0 | 0.2 to 4.0 | 0.3 to 2.0 | 0.6 to 1.4 | 0.8 to 1.2 |
| (b): | | | | | |
| Aqua- | | | | | |
| Style ® | | | | | |
| SH-100 | | | | | |
| (specified | | | | | |
| as solids | | | | | |
| content) | | | | | |
| Carbomer | 0.02 to 3.0 | 0.05 to 2.0 | 0.05 to 1.5 | 0.2 to 1.0 | 0.35 to 0.8 |
| Misc | to 100 | to 100 | to 100 | to 100 | to 100 |

| | Formula | Formula | Formula | Formula | Formula |
|---|---|---|---|---|---|
| | 21 | 22 | 23 | 24 | 25 |
| Vinyl- | 0.05 to 3.0 | 0.1 to 2.5 | 0.2 to 2.0 | 0.3 to 1.6 | 0.4 to 1.4 |
| pyrroli- | | | | | |
| done | | | | | |
| homo- | | | | | |
| polymer | | | | | |
| (a) | | | | | |
| Co- | 0.1 to 5.0 | 0.2 to 4.0 | 0.3 to 2.0 | 0.6 to 1.4 | 0.8 to 1.2 |
| polymer | | | | | |
| (b) | | | | | |
| Carbomer | 0.02 to 3.0 | 0.05 to 2.0 | 0.05 to 1.5 | 0.2 to 1.0 | 0.35 to 0.8 |
| PEG-40 | 0.05 to 1.5 | 0.1 to 1.0 | 0.2 to 0.9 | 0.3 to 0.8 | 0.35 to 0.6 |
| Hydro- | | | | | |
| genated | | | | | |
| Castor Oil | | | | | |
| Misc | to 100 | to 100 | to 100 | to 100 | to 100 |

| | Formula | Formula | Formula | Formula | Formula |
|---|---|---|---|---|---|
| | 21a | 22a | 23a | 24a | 25a |
| Vinyl- | 0.05 to 3.0 | 0.1 to 2.5 | 0.2 to 2.0 | 0.3 to 1.6 | 0.4 to 1.4 |
| pyrrolidone | | | | | |
| homo- | | | | | |
| polymer | | | | | |
| (a) with K | | | | | |
| value of from | | | | | |
| 80 to 100 | | | | | |
| (specified as | | | | | |
| solids | | | | | |
| content) | | | | | |
| Copolymer | 0.1 to 5.0 | 0.2 to 4.0 | 0.3 to 2.0 | 0.6 to 1.4 | 0.8 to 1.2 |
| (b): | | | | | |
| Acrylates | | | | | |
| Copolymer | | | | | |
| (and) Water | | | | | |
| Carbomer | 0.02 to 3.0 | 0.05 to 2.0 | 0.05 to 1.5 | 0.2 to 1.0 | 0.35 to 0.8 |
| PEG-40 | 0.05 to 1.5 | 0.1 to 1.0 | 0.2 to 0.9 | 0.3 to 0.8 | 0.35 to 0.6 |
| Hydro- | | | | | |
| genated | | | | | |
| Castor Oil | | | | | |
| Misc | to 100 | to 100 | to 100 | to 100 | to 100 |

| | Formula | Formula | Formula | Formula | Formula |
|---|---|---|---|---|---|
| | 21b | 22b | 23b | 24b | 25b |
| Vinyl- | 0.05 to 3.0 | 0.1 to 2.5 | 0.2 to 2.0 | 0.3 to 1.6 | 0.4 to 1.4 |
| pyrrolidone | | | | | |
| homo- | | | | | |
| polymer | | | | | |
| (a): | | | | | |
| Luviskol ® | | | | | |
| K 90-20% | | | | | |
| (specified as | | | | | |
| solids | | | | | |
| content) | | | | | |
| Copolymer | 0.1 to 5.0 | 0.2 to 4.0 | 0.3 to 2.0 | 0.6 to 1.4 | 0.8 to 1.2 |
| (b): | | | | | |
| Aqua- | | | | | |
| Style ® | | | | | |
| SH-100 | | | | | |
| (specified | | | | | |
| as solids | | | | | |
| content) | | | | | |
| Carbomer | 0.02 to 3.0 | 0.05 to 2.0 | 0.05 to 1.5 | 0.2 to 1.0 | 0.35 to 0.8 |
| PEG-40 | 0.05 to 1.5 | 0.1 to 1.0 | 0.2 to 0.9 | 0.3 to 0.8 | 0.35 to 0.6 |
| Hydro- | | | | | |
| genated | | | | | |
| Castor Oil | | | | | |
| Misc | to 100 | to 100 | to 100 | to 100 | to 100 |

The entry “Misc” is to be understood in accordance with the invention to mean a cosmetic carrier, in particular water and optionally further usual constituents of styling products.

### EXAMPLES

1. The following styling gel was prepared:

| Composition | 1 | 2 | | |
|---|---|---|---|---|
| Component/raw | % by | % by | | |
| material | weight | weight | INCI name | Manufacturer |
| Water, | 63.355 | 58.24 | | |
| demineralised | | | | |
| Carbomer | 0.39 | | Carbomer | |
| 20000-30000 | | | | |
| mPas (0.2%) | | | | |
| DMDM | 0.1 | 0.1 | DMDM | |
| Hydantoin 55% | | | Hydantoin | |
| Sodium | 0.37 | 0.48 | | |
| hydroxide pearls | | | | |
| Water, | 29.705 | 29.7 | | |
| demineralized, | | | | |
| without H₂O₂ | | | | |
| Luviskol ® | 2 | 7 | Acrylates/ | BASF |
| K90-20% | | | Hydroxyesters | |
| | | | Acrylates | |
| | | | Copolymer | |
| AquaStyle | 3.3 | 3.3 | Acrylates | Ashland |
| SH-100 | | | Copolymer | Inc. |
| | | | (and) Water | |
| Castor oil | 0.4 | 0.4 | PEG-40 | |
| hydrog., 40 EO | | | Hydrogenated | |
| | | | Castor Oil | |
| Perfume TEU-D- | 0.08 | 0.08 | | |
| 41606/6 Nutri | | | | |
| Acai | | | | |
| Polygel HP/ | | 0.6 | Carbomer | 3V |
| Synthalen HP | | | | |
| Methylparabene | | 0.1 | | |
| Total | 100 | 100 | | |

The amounts specified in the table are given in % by weight of the respective raw materials, in relation to the total composition.

The hair gel, when applied to human hair, formed a transparent film with very low tack.

Stiffness:

850 mg of a gel-like test composition was massaged into dry strands of hair (Euro natural hair from the company Kerling, 826500 hair extension glued tightly on one side, total length 150 mm, free length 130 mm, width 20 mm, weight 1.8±0.2 g) using fingers. The strands of hair treated with the examined test composition were straightened using a Teflon rail with a diameter of 20 mm. The prepared strands were then dried and conditioned overnight at 21° C. and 50% relative humidity in an air-conditioned space.

The conditioned strands were carefully removed from the Teflon rail. The flat strands produced were placed onto the 40 mm measuring blocks separated from one another. The 3PB adapter of a universal testing apparatus AMETEK LF Plus from the company AMETEK Precision Instruments Europe GmbH, product group Lloyd was assembled centrally above said measuring blocks. The entire measurement was performed in an air-conditioned space under constant climatic conditions at 21° C. and 50% relative humidity.

In order to create standardized starting conditions, the measurement was started with application of an initial load of 0.05 N. The strands were then pressed at a speed of 500 mm min⁻¹ by 15 mm, wherein the force necessary for this was measured. The characteristic force K at the maximum shaping of 15 mm was then recorded.

This measurement method makes it possible to measure the stiffness of the polymer film and the degree of hold of the hairstyle hold generated, on the basis of the force Fmax as parameter.

10 strands were created and measured per text compositions. Stiffness values ranging from 2.3-3.8 N were obtained, these being arithmetic mean values.

Long-Term Hold:

The composition was examined in respect of its shaping properties by means of a Long Lasting Hold measurement. Standardized hair strands from the company Kerling (product no. 826500 of the “European Natural” hair type, colour 6/0) of a length (Lₘₐₓ) of 220 mm and a weight of 3.0 g were used for this purpose. For preparation, the strands were washed with a 12.5% by weight sodium laureth sulfate solution. The hair strands were dried overnight in a drying oven at 318 K.

The hairs were soaked for 20 min in tepid water and were then dabbed to approximately 50% residual moisture in the hair.

750 mg of the composition were applied to each of the hair strands and massaged in. The hair strands were placed in a Teflon rail, smoothed by means of a steel roll, and dried overnight at 21° C. and 50% humidity.

The hair strands were then clamped at one of their ends in a holding device and stored for a period of six hours at 21° C. and 85% relative humidity. In order to calculate the Long Lasting Hold (LLH), the strand lengths protruding from the holding device before storage (L₀) and after storage (Lₜ) were measured.

The Long Lasting Hold is a measure for the change in length over time of a strand of hair fixed by means of a hair-shaping agent. The higher the LLH value, the lower is the change in length of the strand of hair under the influence of humidity within a specific period of time, and the better is the degree of hold of the hair-shaping agent.

The Long Lasting Hold was calculated in accordance with the following formula.

LLH=1−(Lₜ−L₀/Lmax)

An LLH value of 40-60% was determined (arithmetic mean of the LLH values from 10 test strands).

The gel according to the invention thus demonstrated an extraordinarily good combination of long-term hold and stiffness.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition for temporarily reshaping keratin fibers, said composition comprising:
(a) at least one vinylpyrrolidone homopolymer and,
(b) at least one anionic acrylate copolymer (b) which is constructed at least from the following monomer units: at least one (meth)acrylic acid unit (b1), at least one (meth)acrylic acid ethyl ester unit (b2), and at least one (meth)acrylic acid ester unit (b3) which is different from the (meth)acrylic acid ethyl ester unit (b2) and which comprises a hydrophobic group as ester group.

2. The cosmetic composition according to claim 1, wherein the vinylpyrrolidone homopolymer (a) has a K value (1% by weight solution of PVP, Brookfield at 23° C.) in water of from 20 to 100.

3. The cosmetic composition according to claim 1, wherein the vinylpyrrolidone homopolymer (a) has a K value of from 80 to 100.

4. The cosmetic composition according to claim 1, wherein the vinylpyrrolidone homopolymer (a) has a K value of approximately 90.

5. The cosmetic composition according to claim 1, wherein the anionic acrylate copolymer (b) comprises a (meth)acrylic acid alkyl ester as monomer unit (b3).

6. The cosmetic composition according to claim 1, wherein the composition further comprises at least one thickening agent.

7. The cosmetic composition according to claim 1, wherein the composition further comprises a carbomer as a thickening agent.

8. The cosmetic composition according to claim 1, wherein the vinylpyrrolidone homopolymer (a) has a K value of from 80 to 100, and the anionic acrylate copolymer (b) is one with the INCI name Acrylates Copolymer (and) Water.

9. The cosmetic composition according to claim 1, which, in relation to the total weight of the cosmetic composition, comprises: 0.01 to 3.0% by weight of the vinylpyrrolidone homopolymer (a) and 0.3 to 2.0% by weight of the anionic acrylate copolymer (b).

10. The cosmetic composition according claim 1, comprising, in relation to the total weight of the cosmetic composition: 0.1 to 2.0% by weight of the vinylpyrrolidone homopolymer (a) and 0.8 to 1.4% by weight of the anionic acrylate copolymer (b).

11. A method for temporarily reshaping keratin fibers, comprising, applying the cosmetic composition of claim 1 to keratin fibers.
